(19) United States
(12) Patent Application Publication
(10) Pub. No.: US 2019/0194594 A1
(43) Pub. Date: Jun. 27, 2019

(54) INTEGRATED SYSTEM OF CELL CULTURE AND OPERATION AND OPERATING METHOD THEREOF

(71) Applicants: NINGBO UNIVERSITY, Ningbo (CN); CHINA INNOVATION INSTRUMENT CO., LTD., Ningbo (CN)

(72) Inventors: Zhenzhi SHI, Ningbo (CN); Luhong WEN, Ningbo (CN); Shundi HU, Ningbo (CN); Huanhuan HONG, Ningbo (CN); Jianqin GAN, Ningbo (CN)

(21) Appl. No.: 16/286,552

(22) Filed: Feb. 26, 2019

Related U.S. Application Data
(63) Continuation of application No. PCT/CN2017/119795, filed on Dec. 29, 2017.

(30) Foreign Application Priority Data
Dec. 31, 2016 (CN) 201611268264.0

(51) Int. Cl. C12M 1/34; C12M 1/12; C12M 1/36; C12M 1/02; C12M 1/00; C12M 1/04; C12M 1/107

(52) U.S. Cl. CPC C12M 41/34; C12M 37/00; C12M 37/02; C12M 23/36; C12M 41/20; C12M 23/22; C12M 23/24; C12M 41/48

(57) ABSTRACT

An integrated system of cell culture and operation and a method for controlling the same. The integrated system of cell culture and operation includes a housing, a front side of the housing being provided with operating gloves. The housing has a double-layer structure consisting of an inner layer and an outer layer. An upper wall and a lower wall of the inner layer are both provided with through holes. The outer layer has a gas inlet, a gas outlet and a humidifying port. The integrated system of cell culture and operation further includes: a heating module arranged in an interlayer, and a transport module configured to transport gas and circulate the gas in the inner layer and the interlayer, resulting in low gas consumption.

### CROSS-REFERENCE TO RELATED APPLICATIONS

This application is a continuation of International Application No. PCT/CN2017/119795, filed on Dec. 29, 2017, which claims the benefit of priority to Chinese Application No. 201611268264.0, filed on Dec. 31, 2016. The content of the aforementioned application, including any intervening amendments thereto, is incorporated herein by reference.

### TECHNICAL FIELD

The present invention relates to cell culture, and in particular to an integrated system of cell culture and operation and an working method thereof.

### BACKGROUND OF THE PRESENT INVENTION

Cell culture requires a suitable environment with suitable gas concentration, cleanliness, temperature and humidity (generally, a saturated or high humidity), etc., thereby ensuring safe and normal growth of cells.

Currently, cells are usually cultured in cell culture incubators, while the cell operation is mainly performed on a clean bench or in a biosafety cabinet to avoid contamination. However, such cell culture incubators mainly have the following deficiencies.

1. Contamination may occur during the transport process because the cell culture is separated from the cell operation.

2. The clean bench or biosafety cabinet for cell operation is not provided with gas concentration control and temperature control, so that the consistency of the cell culture environment cannot be ensured, thus affecting the growth of cells.

3. Risk of contamination may be raised in a high-humidity environment due to the formation of condensed water on the inner surface of the cell incubator.

### SUMMARY OF THE PRESENT INVENTION

The present invention provides an integrated system of cell culture and operation with low risk of contamination, low gas consumption and high control efficiency of gas concentration to overcome the deficiencies in the prior art.

An object of the present invention is to provide an integrated system of cell culture and operation, including a housing, wherein a front side of the housing is provided with operating gloves; the housing has a double-layer structure consisting of an inner layer and an outer layer; an upper wall and a lower wall of the inner layer are both provided with through holes; and the outer layer is provided with a gas inlet, a gas outlet and a humidifying port.

The integrated system of cell culture and operation further comprises:

a heating module arranged in an interlayer formed by the inner layer and the outer layer; and

a transport module configured to transport gas and circulate the gas in the inner layer and the interlayer.

Another object of the present invention is to provide an working method of the integrated system of cell culture and operation with low risk of contamination and high control efficiency.

The working method includes the following steps:

(A1) transporting gas by the transport module to flow downward in the inner layer;

(A2) the gas entering the interlayer through a through hole of the lower wall of the inner layer to flow in a lower portion, a side and an upper portion of the interlayer; heating the gas by the heating module;

wherein condensed water is not formed on an inner wall of the inner layer; and

(A3) the gas entering the inner layer through a through hole on the upper wall of the inner layer.

The present invention has the following beneficial effects compared to the prior art.

1. A hot air jacket around the inner layer protects the interlayer. The gas inside the inner layer is heated by air circulation so that the temperature in the interlayer is higher than that inside the inner layer. Therefore, the event of condensed water on an inner wall of the inner layer is prevented to reduce the risk of contamination.

2. The number of cells cultured is effectively increased due to the combination of cell culture and cell operation.

3. The gas loss during the regulation of target gas concentration in the inner layer is reduced, and the efficiency of gas concentration regulation is improved.

### BRIEF DESCRIPTION OF THE DRAWINGS

The disclosure of the present invention will become more apparent with reference to the drawings. It should be understood by those skilled in the art that these drawings are merely used to illustrate the technical solutions of the present invention, but are not intended to limit the scope of the present invention.

FIG. 1 is a schematic diagram of an integrated system of cell culture and operation according to an embodiment of the present invention.

### DETAILED DESCRIPTION OF THE PRESENT INVENTION

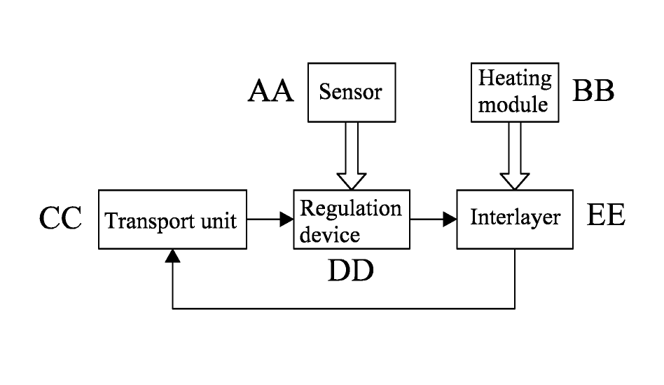

FIG. 1 and the following description describe optional embodiments of the present invention to teach those skilled in the art how to perform and reproduce the present invention. To teach the technical solutions of the present invention, some conventional aspects have been simplified or omitted. It should be understood by those skilled in the art that variants or substitutions derived from these embodiments shall fall within the scope of the present invention. It should be understood by those skilled in the art that the following features can be combined in various ways to form various variants of the present invention. Therefore, the present invention is not limited to the following optional embodiments, but is only defined by the appended claims and equivalents thereof.

### EXAMPLE 1

FIG. 1 schematically shows a structure of an integrated system of cell culture and operation according to an embodiment of the present invention. As shown in FIG. 1, the integrated system of cell culture and operation includes: a housing, a transport module, a plurality of heating modules, a filter, a regulation device, a sensor and a processor.

The housing is provided with a gas inlet, a gas outlet and a humidifying port. The housing has a double-layer structure consisting of an inner layer and an outer layer. An upper wall and a lower wall of the inner layer both are provided with through holes. A front side of the housing is provided with operating gloves.

The transport module, such as a fan, is arranged on the upper wall of the inner layer at a position where the through hole is provided or arranged at a lower side of the through hole to transport the gas in the inner layer to the regulation device so that the gas circulates in the inner layer and an interlayer.

The heating modules such as heating sheets are arranged in the interlayer, for example, on an inner wall of the outer layer and in a side, an upper portion and a lower portion of the interlayer. The heating module at a front side of the interlayer is transparent, which is convenient for observing the inner layer and improves the heating uniformity.

The filter is arranged on a lower portion of the transport module. A projection of the filter in a horizontal plane overlaps with a pattern formed by the surrounding inner wall of the inner layer in the horizontal plane.

The regulation device is in a state of storing or releasing the target gas according to different working parameters. The regulation device is arranged in the upper portion of the interlayer. Here, two regulation devices, including an oxygen molecular sieve and a carbon dioxide molecular sieve, are provided in this embodiment, so that gaseous molecules are adsorbed and desorbed using Pressure Swing Adsorption (PSA) to store or release the target gas.

The sensor is configured to detect contents of oxygen and carbon dioxide in the inner layer and send the detected contents to the processor.

The processor is configured to regulate the working states of the two regulation devices according to the detected contents. If the detected contents are less than a target value, the regulation devices are controlled to be in a state of releasing the target gas; if the detected contents are greater than the target value, the regulation devices are controlled to be in a state of storing the target gas.

This embodiment of the present invention further provides a working method of the integrated system of cell culture and operation, including the following steps.

(A1) The gas is transported by the transport module to flow downward in the inner layer.

(A2) The gas enters the interlayer through a through hole of the lower wall of the inner layer to flow in a lower portion, a side and an upper portion of the interlayer. The gas is then heated by the heating module.

Condensed water is not formed on an inner wall of the inner layer.

(A3) The gas enters the inner layer through a through hole on the upper wall of the inner layer.

A method for controlling the contents of target gases in the inner layer, includes the following steps.

(B1) Contents of target gases, including oxygen and carbon dioxide in the inner layer of the integrated system of cell culture and operation, are detected by a gas sensor. The detected contents are sent to the processor.

(B2) Working states of the regulation devices are controlled by the processor. If the contents of the target gases are to be reduced, proceed to step (B3). If the contents of the target gases are to be increased, proceed to step (B4). If no adjustment is required, proceed to step (B5).

(B3) The regulation devices are controlled to a state of storing the target gas by controlling the parameters of the regulation devices. The gas is transported by the transport module from the lower wall of the inner layer into the interlayer. The gas flows upward to an upper wall of the interlayer along a side wall of the interlayer. The target gases in the gas is stored by the regulating device to finally enter the inner layer from the upper wall of the inner layer. Proceed to step (B1).

(B4) The regulation devices are controlled to a state of releasing the target gas by controlling the parameters of the regulation devices. The gas is transported by the transport module from the lower wall of the inner layer into the interlayer. The gas flows upward flow upward to the upper wall of the interlayer along the side wall of the interlayer. The target gases stored by the regulation devices are released into the gas to finally enter the inner layer from the upper wall of the inner layer. Proceed to step (B1).

(B5) Ends.

In the above process, if the content of the target gas in the inner layer still cannot reach the target value within several cycles, external gas is injected into the housing through a gas inlet thereon. If the content of the target gas in the housing is always less than the target value, gas with the content of the target gas greater than the target value is injected to increase the content of the target gas in the housing. If the content of the target gas in the housing is always greater than the target value, gas with the content of the target gas less than the target value is injected to reduce the content of the target gas in the housing.

### EXAMPLE 2

The present invention provides an integrated system of cell culture and operation which differs from Example 1. In this example:

1. The regulation devices are arranged outside the housing. An input end and an output end of the regulation device is in communication with the sidewall of the interlayer of the double-layer housing, respectively.

2. Additional transport module is provided to transport the gas in the interlayer to the regulation devices.

What is claimed is:

1. An integrated system of cell culture and operation, comprising:
a housing, a front side of the housing being provided with operating gloves;
a heating module arranged in an interlayer formed by the inner layer and the outer layer; and
a transport module configured to transport gas and circulate the gas in the inner layer and the interlayer;
wherein the housing has a double-layer structure consisting of an inner layer and an outer layer, and an upper wall and a lower wall of the inner layer are both provided with through holes, and the outer layer is provided with a gas inlet, a gas outlet and a humidifying port.

2. The integrated system according to claim 1, wherein the heating module is a heating sheet arranged on an inner wall of the outer layer, and the heating module arranged on at a front side of the inner wall of the outer layer is transparent.

3. The integrated system according to claim 1, wherein the heating module is arranged on an inner wall of a side, an upper portion or a lower portion of the outer layer.

4. The integrated system according to claim 1, further comprising:
a filter arranged on a lower portion of the upper wall of the inner layer,
wherein a projection of the filter in a horizontal plane overlaps with a pattern formed by the surrounding inner wall of the inner layer in the horizontal plane.

5. The integrated system according to claim 1, further comprising:
a regulation device that is in a state of storing or releasing a target gas according to different working parameters, wherein an output end of the regulation device is in communication with the interlayer;
a sensor configured to detect content of the target gas and send the detected content to a processor; and
the processor configured to regulate the state of the regulation device according to the detected content.

6. The integrated system according to claim 5, wherein a regulation device is arranged in the interlayer, and the transport module is arranged on a lower side of a through hole on the upper wall of the inner layer.

7. The integrated system according to claim 5, wherein the regulation device is arranged outside the housing.

8. The integrated system according to claim 5, wherein the target gas comprises oxygen and carbon dioxide, and the regulation device is a molecular sieve.

9. A working method of the integrated system according to claim 1, comprising:
(A1) transporting gas by the transport module to flow downward in the inner layer;
(A2) the gas entering the interlayer through a through hole of the lower wall of the inner layer to flow in a lower portion, a side and an upper portion of the interlayer;
heating the gas by the heating module;
wherein condensed water is not formed on an inner wall of the inner layer; and
(A3) the gas entering the inner layer through a through hole on the upper wall of the inner layer.

10. The working method according to claim 9, further comprising:
(B1) detecting, by a gas sensor, content of the target gas in the integrated system of cell culture and operation, and sending the detected content to the processor;
(B2) controlling, by the processor, the state of the regulation device according to the detected content;
if the content of the target gas is to be reduced, proceeding to step (B3);
if the content of the target gas is to be increased, proceeding to step (B4);
if no adjustment is required, proceeding to step (B5);
(B3) controlling the regulation device to a state of storing the target gas by controlling parameters of the regulation device; storing the target gas when the gas in the housing flows through the regulation device and transporting the gas back to the housing after the gas is processed; proceeding to step (B1);
(B4) controlling the regulation device to a state of releasing the target gas by controlling parameters of the regulation device; releasing the target gas when the gas in the housing flows through the regulation device; transporting the gas back to the housing after the gas is processed; proceeding to step (B1); and
(B5) ending.
